(19) United States
(12) Patent Application Publication
(10) Pub. No.: US 2005/0234280 A1
(43) Pub. Date: Oct. 20, 2005

(54) Material for a thin and low-conductive funtional layer for an oled and production method therefor

(76) Inventors: Georg Wittmann, Herzogenaurach (DE); Karsten Heuser, Erlangen (DE); Jan Birnstock, Dresden (DE); Andreas Kanitz, Hochstadt (DE); Jasmin Worle, Fuerth (DE)

Correspondence Address: FISH & RICHARDSON PC, P.O. BOX 1022, MINNEAPOLIS, MN, 55440-1022, US

(21) Appl. No.: 10/517,892

(22) PCT Filed: Jun. 10, 2003
(86) PCT No.: PCT/DE03/01912

§ 371 Date: Jun. 23, 2005

(30) Foreign Application Priority Data
Jun. 14, 2002 (DE) 102 26 616.6

(51) Int. Cl. C07C 1/00; B05D 5/12

(52) U.S. Cl. 585/639; 427/66; 427/240; 427/355; 427/421.1

(57) ABSTRACT

The invention relates to a material for applying thin organic layers having a conductivity that can be set in a defined manner. The material comprises at least one mixture consisting of two different fractions of a functional polymer, preferably in a solvent, and is applied, for example, in the form of a thin and low-conductive functional layer of an organic light-emitting diode (OLED) by means of different application techniques.

This invention relates to a material for a thin and low-conductivity functional layer of an organic light-emitting diode (OLED), particularly for a low-conductivity polymer film that is suitable for use as an injection and/or barrier film of an OLED.

Materials for injection layers for OLEDs are known, for example consisting of PEDOT-PSS (poly[3,4-ethylenedioxythiophene]-poly[styrenesulfonate]) or PANI (polyaniline)-PSS. These materials provide injection layers as thin films of the particular functional polymer.

However, when preparing the injection layer, very precisely set conductivities are advised. For example, an organic passive matrix display (PMD) based on π-conjugated polymers has a layer of the conductive polymer mixture PEDOT:PSS (poly[3,4-ethylenedioxythiophene]:poly[4-styrenesulfonic acid]) that typically has a thickness of about 100 nm and is preferably structured. This layer must not have too high a conductivity, since otherwise there is “cross-talk” between the individual image points of the display. However, if the conductivity is too low, the efficiency of the display becomes lower since injection and current transport are severely impaired and thus the entire component becomes uneconomical.

Methods for varying the conductivity of the polymer mixture to some extent are in fact known, but of course its properties are also thereby changed, especially the rheological properties of the polymer dispersion or solution such as surface tension and/or viscosity, for example. However, this is a drawback to the known methods, since one of the fundamental problems when handling conductive polymers is how they are applied to substrates to be coated. Different coating methods are customary by which the polymer is applied by wet chemistry from a solution or dispersion, for example spin coating, printing processes such as screen printing, ink jet printing, or flexo printing, as well as blade processes. It is common to all of the methods that the viscosity and concentration of the solution or dispersion play a critical role for processing a homogeneous layer and/or a defined thickness.

Therefore, it is the purpose of this invention to make available a material for a low-conductivity polymer film in which the conductivity of the polymer film to be produced can be set selectively while retaining the solution and/or dispersion properties optimal for the coating method.

It is the general known background of the invention that a mixture of different fractions of a single functional polymer has conductivity that can be adjusted by the mixing ratio, with the rheological properties of such a mixture being unimpaired by the ratio of the fractions in the mixture.

The object of this invention is a material for forming a thin film whose conductivity can be set in the range of 10⁻⁴ S/cm to 10⁻⁶ S/cm and whose thickness is between 10 and 300 nm, with the material comprising a mixture of at least two different fractions of a functional polymer, namely a first fraction that is based on a dispersion of the functional polymer in a first solvent in which the functional polymer is at least partly dispersed, and a second fraction of functional polymer that is based on a true solution of the functional polymer in a second solvent, with the two fractions being processed, dispersed, and/or dissolved together, with the ability to set the conductivity of the thin film composed of this material by the ratio in which the at least two fractions are mixed. Also an object of the invention is a method for preparing a material for forming a thin film in which a mixture of two different fractions of a functional polymer is combined, in a solvent as the case may be.

According to an embodiment of the method, high-boiling solvent is added for the purpose to a dispersion of the functional polymer and a solution of the functional polymer, and the lower-boiling solvents are then removed by distillation so that ultimately the different fractions of functional polymer in the high-boiling solvent essentially constitute the material. One embodiment of the method provides that the high-boiling solvent in each case is added in the same amount as the fraction that is present.

According to a beneficial embodiment, the material is essentially free of the solvent and/or dispersing agent of the underlying fractions and/or comprises an additional, third solvent. The material can contain any other admixtures and additives that are customary and/or reasonable for these types of materials, such as defoamers or wetting agents, etc.

The material pursuant to the invention for forming a functional layer of an OLED is called “material” in the present case.

According to another embodiment, the two fractions are both in dry form before the dispersion/dissolving.

The two fractions designate two modifications, or two presumably different states of a substance.

According to a beneficial embodiment, the functional polymer is PEDOT or PANI.

According to a beneficial embodiment, the functional polymer is a copolymer or blend that includes PSS polystyrenesulfonate as anions.

According to an embodiment, the first solvent is water or another component with high polarity in which the functional polymer is essentially insoluble.

According to an embodiment, the second solvent is ethanol or another low-boiling, polar solvent, preferably a polar protic one that can develop hydrogen bridge bonds.

The term “low-boiling” here means solvents that have boiling points up to 150° C.

According to an embodiment, the third solvent is different from the first and/or from the second solvent.

According to an advantageous embodiment, ethylene glycol or another alcohol is used, particularly including mixtures of several alcohols, and/or alcohols with a carbon content from C4 to C10, branched and unbranched, and also polyfunctional alcohols, and/or mixtures thereof, as well as mixtures with water, and with special preference glycol and glycerol.

In this context, a thin film composed of functional polymer that is positioned between the anode and the emitter layer of an OLED and is usually structured, is called an injection layer. This layer increases the efficiency and lifetime of the electrodes, especially of an ITO anode.

The term “solution” is used when individual polymer particles are surrounded essentially by solvent molecules, and it is contrasted with the term “dispersion” that designates the state in which individual polymer particles conglomerate and form clusters, for example, but which do not precipitate or settle out, but are essentially dispersed, and form no precipitate or large solid agglomerates. Whether a component here is called a solvent or a dispersing agent depends on how the particular functional polymer in question behaves in this medium. The conditions prevailing during preparation, storage, and/or processing must be taken into consideration in each case.

The term “organic material” or “functional material” or “functional polymer” here includes all types of organic, organometallic, and/or organic-inorganic synthetics (hybrids), particularly those that are called “plastics” in English, for example. They involve all types of substances with the exception of semiconductors that form classical diodes (germanium, silicon), and the typical metallic conductors. Accordingly, no restriction in the dogmatic sense to organic material as a carbon-containing material is intended, but instead the broad use of silicones, for example,.is in mind. Furthermore, the term is intended not to be subject to limitation with respect to the molecular size, particularly to polymeric and/or oligomeric materials, but the use of “small molecules” is also definitely possible. The “polymer” part of “functional polymer” is historically derived and to that extent says nothing about the presence of an actual polymeric compound and nothing about whether or not a polymer blend or a copolymer is involved.

A substance that is essentially free of solvent is called here a dry substance.

The invention will also be described below with reference to an example of preparation:

The conductivity is modified by many orders of magnitude here for the first time without changing the solvent environment. For example, a mixture is used of two different PEDOT solutions (both with the same solvent, e.g. ethylene glycol) that have different conductivities because of their prior histories (one solution is prepared from a water-based solution, and the other from an ethanol-based solution). The solution that was obtained from water-based PEDOT (WPEDOT) has a specific resistance of 10\hat{ }2 Ωcm, and that obtained from ethanol-based PEDOT (EPEDOT) has one of 10\hat{ }7 Ωcm.

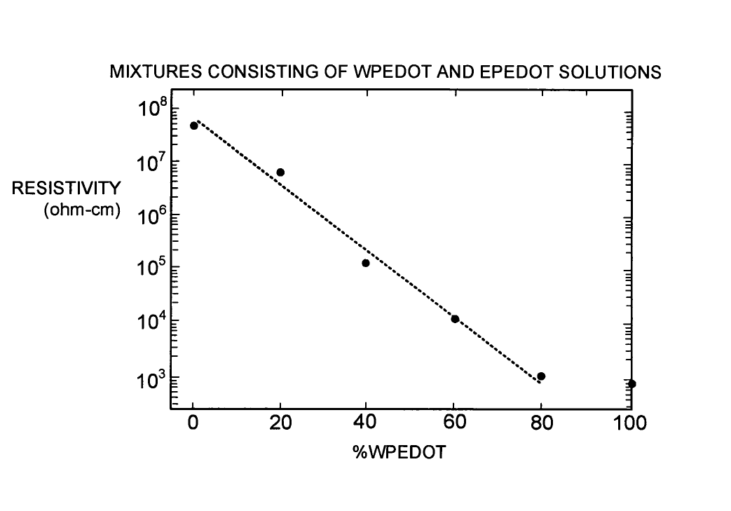

To prepare the starting materials WPEDOT and EPEDOT, the same volume of ethylene glycol is added to the original solutions, which are sold commercially by HC Starck and others, and the original solvent is then distilled off in a rotary evaporator. Since ethylene glycol can be distilled only at 200° C., a pure solution of PEDOT in glycol then remains. Since the original materials WPEDOT and EPEDOT are of different natures, in the case of WPEDOT the conductivity is drastically reduced by replacing the water with ethylene glycol, which lies in the dispersive character of the WPEDOT. In the case of EPEDOT, which is an actual solution, the conductivity is not changed by the replacement of ethanol by ethylene glycol. Thus, two glycolic PEDOT variations are formed with conductivities that differ by 5 orders of magnitude. Any conductivity between these can then be set by mixing (blending) the two solutions (see FIG. 1).

The problem described initially of selectively fine-tuning the conductivity of the polymer film over many orders of magnitude while retaining the optimal solution and dispersion properties for the coating process should be solved by the present invention. This invention makes it possible to apply a polymer film whose conductivity can be selected at will over a broad range, structured or continuously, to a substrate, with high resolution, by an economical coating method such as screen printing, for example. This is possible since the conductivity of the polymer is varied by different mixing ratios of the first and second fractions of the functional polymer and/or by the choice of the third solvent, without adding additives. Thus the surface tension and viscosity remain unchanged and the printability of the polymer is retained.

The invention relates to a material for a functional layer of an organic light-emitting diode (OLED), particularly for a low-conductivity polymer film that is suitable for use as an injection, planarizing, and/or barrier layer of an organic light-emitting diode (OLED). The material comprises at least one mixture consisting of two different fractions of a functional polymer, preferably in a solvent.

1. Material for forming a thin film whose conductivity can be set in the range of 10⁻⁴ S/cm to 10⁻⁶ S/cm and whose thickness is between 10 and 300 nm, with the material comprising a mixture of at least two different fractions of a functional polymer, namely a first fraction that is based on a dispersion of the functional polymer in a first solvent in which the functional polymer is at least partly dispersed, and a second fraction of functional polymer that is based on a true solution of the functional polymer in a second solvent, with the two fractions being processed, dispersed, and/or dissolved together, with the ability to set the conductivity of the thin film composed of this material by the ratio in which the at least two fractions are mixed.

2. Material of claim 1 that contains an additional third solvent.

3. Material of claim 1 that is essentially free of the first and/or second solvent and/or dispersing agent of the underlying fractions.

4. Material of claim 1 wherein the functional polymer comprises PEDOT or PANI.

5. Material of claim 1 wherein the functional polymer is present as a copolymer or blend that includes PSS.

6. Material of claim 1 wherein the first solvent includes water or another component with high polarity in which the functional polymer is essentially insoluble.

7. Material of claim 1 wherein the second solvent is ethanol or another low-boiling polar solvent, preferably a polar protic solvent that can develop hydrogen bridge bonds.

8. Material of claim 2 wherein the third solvent is different from the first and/or second solvent.

9. Material of claim 1 wherein ethylene glycol or another alcohol is used as a third solvent, especially including mixtures of several alcohols, and/or alcohols with a carbon content from C4 to C10, branched and unbranched, and also polyfunctional alcohols or mixtures thereof, and mixtures with water, with special preference glycol and glycerol.

10. Method for preparing a material for a functional layer with a conductivity in the range of 10⁻⁴ S/cm to 10⁻⁶ S/cm and a maximum thickness of 100 nm, in which a mixture consisting of two different fractions of a functional polymer is combined, in a solvent as the case may be.

11. Method of claim 10 wherein a third, high-boiling solvent is added to a dispersion of the functional polymer as the first fraction and a solution of the functional polymer as the second fraction, and the lower-boiling solvents are then removed by distillation so that ultimately the different fractions of functional polymer without their own solvent essentially constitute the material in the third, high-boiling solvent.

12. Method of claim 10 wherein the high-boiling solvent is added in the same amount as that of each fraction that is present.

13. Preparation of a thin film for an OLED with conductivity that can be preset, consisting of a material of claim 1, with one of the following techniques being used: spin coating, screen printing, offset printing, flexo printing, spray coating, roller coating, ink jet printing, stencil printing, or blade coating.

14. A method comprising using the material of claim 1 in OLEDs.

15. Material of claim 2 that is essentially free of the first and/or second solvent and/or dispersing agent of the underlying fractions.

16. Material of claim 8 wherein the first solvent includes water or another component with high polarity in which the functional polymer is essentially insoluble.

17. Material of claim 8 wherein the second solvent is ethanol or another low-boiling polar solvent, preferably a polar protic solvent that can develop hydrogen bridge bonds.

18. Material of claim 8 wherein ethylene glycol or another alcohol is used as the third solvent, especially including mixtures of several alcohols, and/or alcohols with a carbon content from C4 to C10, branched and unbranched, and also polyfunctional alcohols or mixtures thereof, and mixtures with water, with special preference glycol and glycerol.

19. Material of claim 18 wherein the first solvent includes water or another component with high polarity in which the functional polymer is essentially insoluble, and the second solvent is ethanol or another low-boiling polar solvent, preferably a polar protic solvent that can develop hydrogen bridge bonds.

20. Method of claim 11 wherein the high-boiling solvent is added in the same amount as that of each fraction that is present.
